(19) United States
(12) Patent Application Publication
(10) Pub. No.: US 2016/0084948 A1
(43) Pub. Date: Mar. 24, 2016

(54) SYSTEMS, METHODS AND COMPUTER PROGRAM PRODUCTS FOR DOPPLER SPATIAL COHERENCE IMAGING

(71) Applicant: Duke University, Durham, NC (US)

(72) Inventors: Jeremy Dahl, Durham, NC (US); Gregg E. Trahey, Durham, NC (US)

(21) Appl. No.: 14/891,039

(22) PCT Filed: May 28, 2014
(86) PCT No.: PCT/US14/39779

§ 371 Date: Nov. 13, 2015

Related U.S. Application Data
(60) Provisional application No. 61/827,815, filed on May 28, 2013.

(51) Int. Cl. G01S 7/52; A61B 8/08; A61B 8/06; A61B 8/14; A61B 8/00

(52) U.S. Cl. CPC G01S 7/52036; A61B 8/14; A61B 8/587; A61B 8/06; A61B 8/5269

(57) ABSTRACT

Methods, systems and computer program products for imaging fluid in a sample include acquiring a plurality of echo signals for a region of interest in the sample from a plurality of ultrasound transducer elements in an ultrasound transducer array; applying a stationary echo cancellation to the plurality of echo signals to reduce or remove echo signals that are associated with stationary and/or slower moving features to provide a corresponding plurality of filtered signals; extracting coherence information from the plurality of filtered signals; and imaging the region of interest in response to the coherence information from the plurality of filtered signals.

### RELATED APPLICATIONS

This application claims priority to U.S. Provisional Application Ser. No. 61/827,815, filed May 28, 2013, the disclosure of which is hereby incorporated by reference in its entirety.

### FIELD OF THE INVENTION

The present invention relates to Doppler imaging, and in particular, to systems, methods and computer program products for imaging a region using spatial coherence information.

### BACKGROUND

Conventional power Doppler (PD) relies on an ensemble of radio-frequency (RF) traces to determine if a fluid flow is present. A wall filter (or highpass filter) is applied to the summed or ensemble of RF signals to attenuate any stationary and slow moving signals. A PD image pixel is generated by summing the power, or squared magnitude, of the complex signal across the filtered ensemble.

As illustrated in FIG. 1, an ultrasound system 10 includes a controller 20, a B-mode flow processor 30 and an ultrasound transducer array 40, a beamformer 70 and a display 80. The ultrasound transducer array 40 may include a plurality of array elements 42. The array elements 42 are configured to transmit and receive ultrasound signals to/from a tissue medium 60, which may include a target region 62. As illustrated, echo signals 50 resulting from the transmit signals propagate from the target region 62 to the elements 42 of the array 40. In a conventional PD mode, the array elements 42 send/receive ultrasound signals according to a time delay at times T₁ through Tₙ. The beamformer 70 receives time-delayed signals 72 and sums the signals 72 together at a summation unit 74. The summed signals are received by the B-mode flow processor 30, and a wall or highpass filter is applied to the summed RF signals. An image is generated by summing the power, or squared magnitude of the complex signal and displayed on the display 80 according to conventional B-mode imaging techniques. Power Doppler imaging may be used to detect fluid flow in many applications.

The output signal in PD imaging is dependent on the energy of the Doppler signal. If the backscatter of the blood signal is stronger than the background thermal noise, then the blood flow can be differentiated from the thermal noise by using a threshold. However, if the flow is sufficiently slow such that the blood signal is attenuated by the wall filter, it may be more difficult to differentiate blood flow from noise. In addition, slowly moving tissue or reverberation clutter that can pass through the wall filter may degrade the PD image because it appears similar to slowly moving blood.

### SUMMARY OF EMBODIMENTS OF THE INVENTION

In some embodiments, a method for imaging fluid in a sample includes: acquiring a plurality of echo signals for a region of interest in the sample from a plurality of ultrasound transducer elements in an ultrasound transducer array; applying a stationary echo cancellation to the plurality of echo signals to reduce or remove echo signals that are associated with stationary and/or slower moving features to provide a corresponding plurality of filtered signals; extracting coherence information from the plurality of filtered signals; and imaging the region of interest in response to the coherence information from the plurality of filtered signals.

In some embodiments, the echo signals are responsive to time-delayed interrogation signals from the ultrasound transducer elements.

In some embodiments, the echo signals are responsive to a laser acoustic excitation.

In some embodiments, applying a stationary echo cancellation includes applying a highpass or eigendecomposition filter to the plurality of time-delayed echo signals.

In some embodiments, extracting coherence information from the plurality of filtered signals includes computing correlation coefficients, covariance normalization, cross correlation coefficients, and/or cosine of the phase difference of filtered signals for the plurality of filtered signals.

In some embodiments, the method further includes storing the plurality of time-delayed echo signals and then applying the stationary echo cancellation to the plurality of time-delayed echo signals to reduce or remove echo signals that are associated with stationary and/or slower moving features to provide a corresponding plurality of filtered signals.

In some embodiments, imaging the region of interest in response to the coherence information from the plurality of filtered signals includes overlaying the coherence information on an image.

In some embodiments, a system for imaging fluid in a sample includes: an ultrasound transducer array having a plurality of ultrasound transducer elements. The ultrasound transducer array is configured to acquire a plurality of echo signals for a region of interest in the sample from the plurality of ultrasound transducer elements in the ultrasound transducer array. A controller includes a stationary echo cancellation unit configured to apply a stationary echo cancellation to the plurality of echo signals to reduce or remove echo signals that are associated with stationary and/or slower moving features to provide a corresponding plurality of filtered signals, and a coherence information unit configured to extract coherence information from the plurality of filtered signals and to image the region of interest in response to the coherence information from the plurality of filtered signals.

In some embodiments, a computer program product for imaging fluid in a sample includes a non-transient computer readable medium having computer readable program code embodied therein. The computer readable program code includes: computer readable program code configured to acquire a plurality of echo signals for a region of interest in the sample from a plurality of ultrasound transducer elements in an ultrasound transducer array; computer readable program code configured to apply a stationary echo cancellation to the plurality of echo signals to reduce or remove echo signals that are associated with stationary and/or slower moving features to provide a corresponding plurality of filtered signals; computer readable program code configured to extract coherence information from the plurality of filtered signals; and computer readable program code configured to image the region of interest in response to the coherence information from the plurality of filtered signals.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a schematic diagram of a conventional Doppler imaging system.

FIG. 2 is a schematic diagram of a spatial coherence imaging system according to some embodiments.

FIG. 3 is a schematic diagram of a spatial coherence imaging system with a laser excitation device according to some embodiments.

FIG. 4 is a flowchart illustrating operations according to some embodiments.

FIG. 5 is a graph of the energy ratio for Power Doppler (PD) (dashed lines) and Spatial-Coherence Power Doppler (SPD) (solid lines) as a function of channel noise for ensemble lengths of 3, 5 and 15. The energy ratio is greater for the PSD images than for the PD images over the range of noise values.

FIG. 6 is a B-mode image of a flow phantom showing a 4 mm vessel at approximately 4.75 cm depth.

FIG. 7 is a graph of the PD and SPD profiles of the center image line from a flow phantom according to some embodiments in which the vessel is observed at a 4.75 depth. The PD graph demonstrates thermal noise that increases with increasing depth, and the SPD graph demonstrates a reduced sensitivity to thermal noise.

FIG. 8A is a set of PD images as a function of ensemble length illustrating that flow detection is improved with a larger ensemble.

FIG. 8B is a set of SPD images according to some embodiments in which flow may be observed with reduced ensemble sizes.

FIG. 9A is an in vivo PD image of a thyroid.

FIG. 9B is an in vivo SPD image of a thyroid according to some embodiments.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system.

The computer-usable or computer-readable medium may be, for example but not limited to, an electronic, optical, electromagnetic, infrared, or semiconductor system, apparatus, or device. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM).

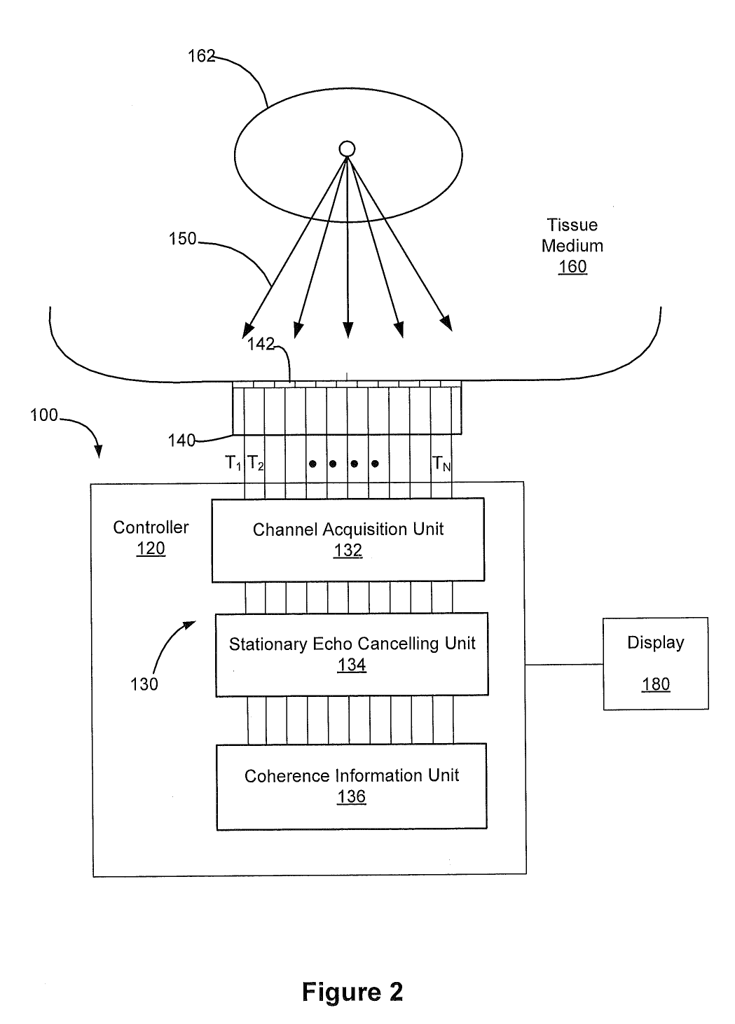

As illustrated in FIG. 2, an ultrasound system 100 includes a controller 120, a signal beamformer and analyzing processor 130, an ultrasound transducer array 140, and a display 180. The ultrasound transducer array 140 may include a plurality of array elements 142. The array elements 142 are configured to transmit and receive ultrasound signals to/from a tissue medium 160, which may include a target region 162. As illustrated, echo signals 150 are transmitted from the target region 162 to the elements 142 of the array 140. In an ultrasound Doppler mode, the array elements 142 send/receive ultrasound signals according to a time delay at times T₁ through Tₙ.

The ultrasound transducer array 140 may be a one-dimensional array configured to generate M-mode and/or two-dimensional images or the ultrasound transducer array 140 may be a two-dimensional array configured to generate M-mode, two-dimensional and/or three-dimensional images.

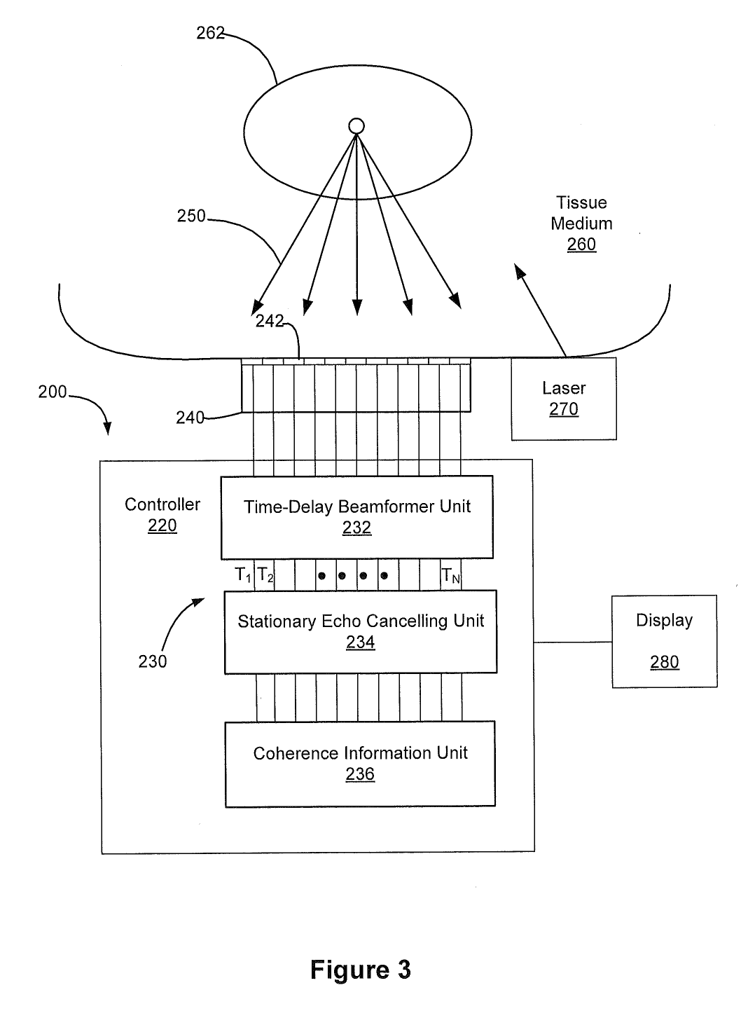

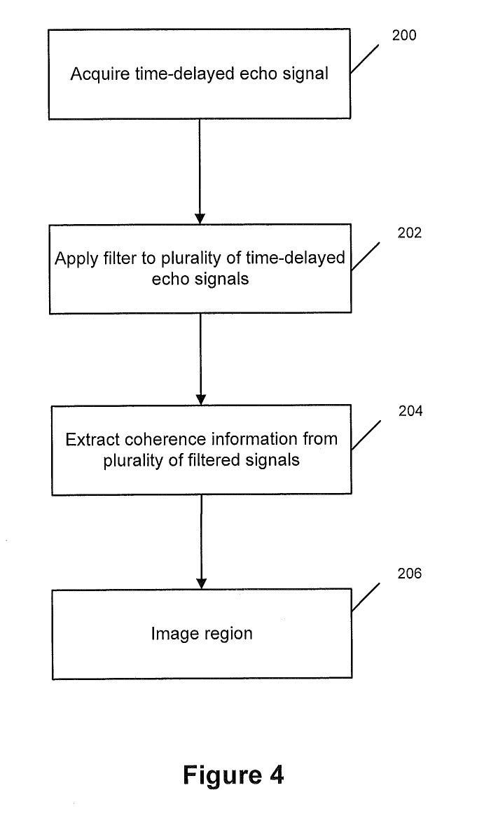

With reference to FIGS. 2 and 4, the signal analyzing processor 130 may include a channel acquisition unit 132 that is configured to acquire and/or store the individual, time-delayed signals from the array elements 142 corresponding to times times T₁ through Tₙ, a stationary echo cancelling unit 134 and a coherence information unit 136. Accordingly, a plurality of time-delayed echo signals for the target region 162 may be acquired from a plurality of ultrasound transducer elements in an ultrasound transducer array (FIG. 3; Block 200). The signals may be time-delayed signals from an ultrasound transducer 140 in which the interrogation pulses (and corresponding echo signals) are time-delayed, e.g., as may be typical in Doppler ultrasound. A stationary echo cancellation, such as a wall filter (highpass filter), may be applied to the plurality of time-delayed echo signals to reduce or remove echo signals that are associated with stationary and/or slower moving features to provide a corresponding plurality of filtered signals (FIG. 3; Block 202). The filtered signals are received by the coherence information unit 136, and coherence information is extracted from the filtered signals (FIG. 3; Block 204). The target region 162 may be imaged in response to the coherence information from the plurality of filtered signals and displayed on the display 180 (FIG. 3; Block 206). The target region may be imaged by overlaying the coherence information on another image, such as a B-Mode image, M-Mode image and/or an Acoustic Radiation Force Impulse (ARFI) image.

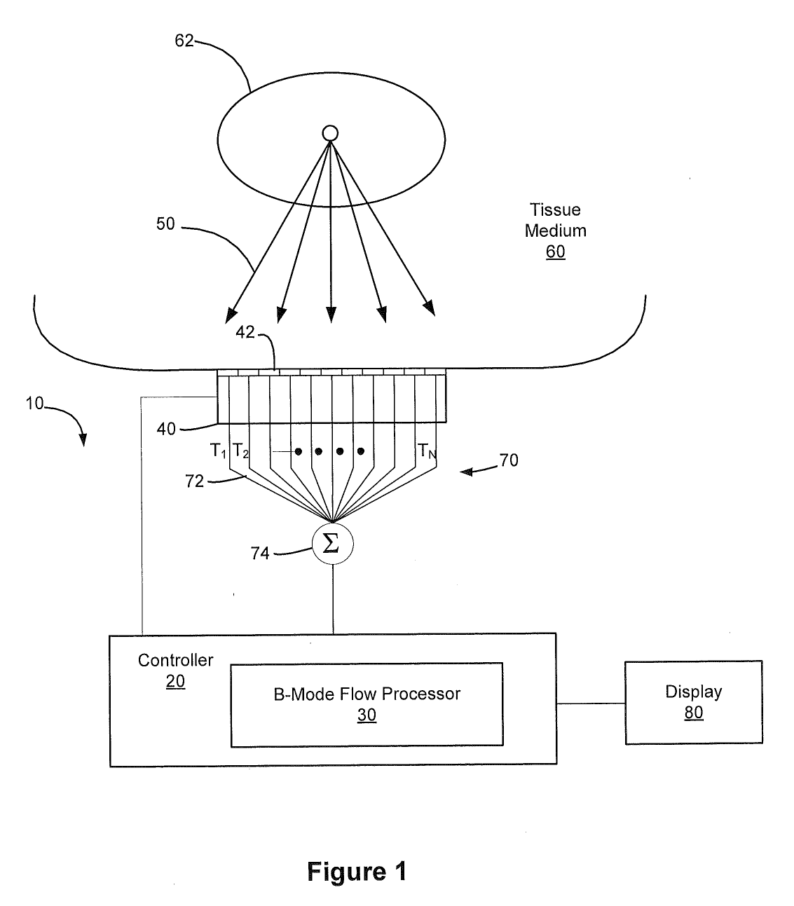

Accordingly, the individual RF signals from the transducer array elements 142 may be stored in a memory and/or filtered so that coherence information may be extracted rather than summing the RF signals prior to processing as shown, for example, in FIG. 1. The RF signals are not fully beamformed (e.g., summed) as is typically performed in conventional Doppler imaging. The filter, such as the stationary echo cancelling unit 134, is therefore applied in a direction across the ensemble for each channel individually. After filtering across the ensemble or individual RF channel signals, the coherence information is extracted for each vector in the ensemble. The resulting coherence Doppler images may then be combined by various techniques, including summing or averaging, and displayed. In some embodiments, the application of ensemble filtering of individual Doppler RF signals may preserve coherence information.

Although embodiments according to the invention are described with respect to the ultrasound transducer array 140 being configured to transmit and receive ultrasound signals to/from the tissue medium 160, it should be understood that other excitation techniques, such as photoacoustic techniques may be used. In photoacoustic imaging, a laser is used to excite acoustical signals in the tissue medium. As illustrated in FIG. 3, an ultrasound system 200 includes a controller 220, a signal analyzing processor 230, an ultrasound transducer array 240, a laser 270 and a display 180. The ultrasound transducer array 240 may include a plurality of array elements 242. The array elements 242 may be configured to transmit and receive ultrasound signals to/from a tissue medium 260, which may include a target region 262. As illustrated, the laser 270 excites the acoustic signals in the tissue medium 260, and the echo signals 250 are transmitted from the target region 262 to the elements 242 of the array 240.

Although embodiments according to the invention are described with respect to time-delayed ultrasound signals, it should be understood that the plurality of echo signals may be beamformed or partially beamformed echo signals received from a location in the region of interest. Any suitable beamforming technique may be used, including beamforming techniques known to those of skill in the art, such as a delay-and-sum beamformer and/or a frequency domain beamformer. In some embodiments, harmonic signals may be extracted from the echo signals, for example, using a pulse inversion technique or harmonic filter, and coherence information may be further extracted from the resulting harmonic signals. Contrast agents may also be added to the fluid being imaged. In a contrast agent imaging mode, for example, pulse inversion techniques or other techniques known to those of skill in the art may be used to reduce a signal of the tissue that does not include the contrast agent, e.g., the tissue surrounding blood or other fluid being imaged. Coherence information may then be extracted from the resulting contrast image mode signals. Examples of contrast agents include microbubbles and nano-emulsions.

In particular embodiments, a spatial coherence function is computed to extract the coherence information and is integrated over the short-lag region, and the resulting value is displayed. As illustrated, the signal analyzing processor 230 includes a time-delay beamformer 232 that may introduce a time delay (e.g., a time delay at times T₁ through Tₙ) to the signals from the transducer elements 242, a stationary echo cancelling unit 234 that is configured to filter the time-delayed signals to reduce or remove stationary features, and a coherence information unit 236 that is configured to extract the coherence information as described herein. The ultrasound transducer array 240 may be a one-dimensional array configured to generate M-mode and two-dimensional images or the ultrasound transducer array 240 may be a two-dimensional array configured to generate M-mode, two-dimensional and/or three-dimensional images.

With reference to FIGS. 2-4, any suitable stationary echo cancellation technique may be used to reduce or remove echo signals from stationary and/or slower moving features, including a wall filter, such as a highpass or eigen-decomposition filter. Moreover, any suitable technique for the extraction of coherence information may be used, including computing correlation coefficients, covariance normalization, cross correlation coefficients and/or cosine of phase differences. In some embodiments, a short-lag spatial coherence (SLSC) technique may be used to extract the coherence information and/or image the target region 162/262. Imaging using SLSC techniques are discussed in International Application Publication No. 2011/123529, the disclosure of which is hereby incorporated by reference in its entirety. In some embodiments, coherence information such as the coherence factor (see, e.g., U.S. Pat. No. 6,071,240), the generalized coherence factor (see, e.g., U.S. Patent Application Publication No. 2005/0228279), and the phase and sign coherence factors (see, e.g., Camacho et al., “Phase Coherence Imaging,” IEEE Transactions on Ultrasonics, Ferroelectrics, and Frequency Control, vol. 56, no. 5 (2009)) can be utilized. Because the backscattered flow signal may have the same or similar coherence properties as tissue, the resulting coherence information may yield a bright signal from the flowing fluid. Thermal noise and any reverberation clutter or other acoustical noise may be suppressed because of the corresponding low coherence properties.

In some embodiments, an image pixel is created using the power or magnitude of coherence vectors, such as SLSC vectors, across the ensemble of individual RF signals as follows where V_{slsc} is the SLSC image magnitude.

$$SPD = {\sum }_{n = 1}^{N} {V}_{slsc}^{2} ( n ) \\ or \\ SPD = {\sum }_{n = 1}^{N} {V}_{slsc} ( n ) \qquad ( 1 )$$

Embodiments according to the present invention will now be described with respect to the following non-limiting examples.

Simulations

Simulations of the cross-section of a 2 mm vessel were performed using Field II (J. A. Jensen, “Field: A program for simulating ultrasound systems,” Med. Biol. Eng. Comp. col. 10th Nordic-Baltic Conference on Biomedical Imaging, vol. 4, no. 1, pp. 351-353)(1996)). The blood signal within the vessel was generated by scatterers at 20 dB below the surround tissue scatterers and traveling at a maximum rate of 5 cm/s with a parabolic profile. The scatterers for both the tissue and blood had a density of 10 scatterers per resolution volume. The simulations were performed with a 128-element 7.5 MHz transducer having a pitch of 0.3 mm. The transmitting pulse had a fractional bandwidth of 30% and used an F/2 transmit beam focused at 2 cm.

Simulated channel signals were acquired for a field-of-view of 5 mm with 0.1 mm beam spacing (for a total of 50 locations). For each location, an ensemble of 15 beams was acquired at a pulse repetition frequency (f_{prf} of 2 kHz). This ensemble was resampled at 666.7 Hz and 400 Hz to yield additional ensemble sizes of 5 and 3, respectively. Thermal noise was incorporated into the simulated channel signals by adding white noise of −40 to 15 dB relative to the blood signal. A two-tap, highpass, Butterworth IIR filter with projection initialization and a 10 Hz cutoff frequency was used to filter the ensembles. Power Doppler (PD) and Spatial-Coherence Power Doppler (SPD) images as described with respect to FIG. 2 were formed over the range of thermal noise and ensemble sizes using a kernel size (K) equivalent to 3λ. The quality of the PD and SPD images was determined using the ratio of the integrated energy within the vessel to the integrated energy of a model vessel. For the discretized images, this is defined as

$$ER = \frac{\sum {\sum }_{A} P ( x , z )}{\sum {\sum }_{A} M ( x , z )} \qquad ( 2 )$$

where P(x, z) is the PD or SPD image in decibels, A is the area of the vessel cross-section, and M(x, z) is a model profile of the power in the vessel. The model can be chosen to match any desired or ideal PD or SPD profile. In the following measurements, the model vessel was chosen to have a flat profile with PD or SPD signal equal to the maximum value within the PD or SPD image, respectively. The integrated energy of P(x, z) and M(x, z) are calculated after shifting the background noise such that its mean is 0 dB.

Phantom and In Vivo Experiments

Individual channel signals from a flow phantom and in vivo thyroid were acquired with an ATL L12-5 transducer and a Verasonics V-1 system (Verasonics, Inc., Redmond, Wash.). The flow phantom contained a 4 mm vessel, for which a cornstarch and water mixture was used to generate scattering within the vessel. A continuous-flow pump was used to circulate the fluid at 5±0.5 cm/s. The transducer transmitted 6 cycle pulses with a frequency of 5 MHz and an F/2 configuration. 128 channel signals were acquired for 50 locations with an ensemble size of 15 at an f_{prf} of 1 kHz. In addition, a corresponding B-mode image having 129 lateral locations was also acquired. A two-tap, highpass, Butterworth IIR filter with projection initialization and a 25 Hz cutoff frequency was used to filter the ensemble signals. The ensemble size for the phantom experiment was varied in order to compare the effect of low ensemble sizes on the resulting PD and SPD images. The vessels observed in the in vivo thyroid were confirmed by imaging with a Siemens Acuson S2000 ultrasound scanner (Siemens Medical Solutions USA, Inc., Issaquah, Wash.).

Results

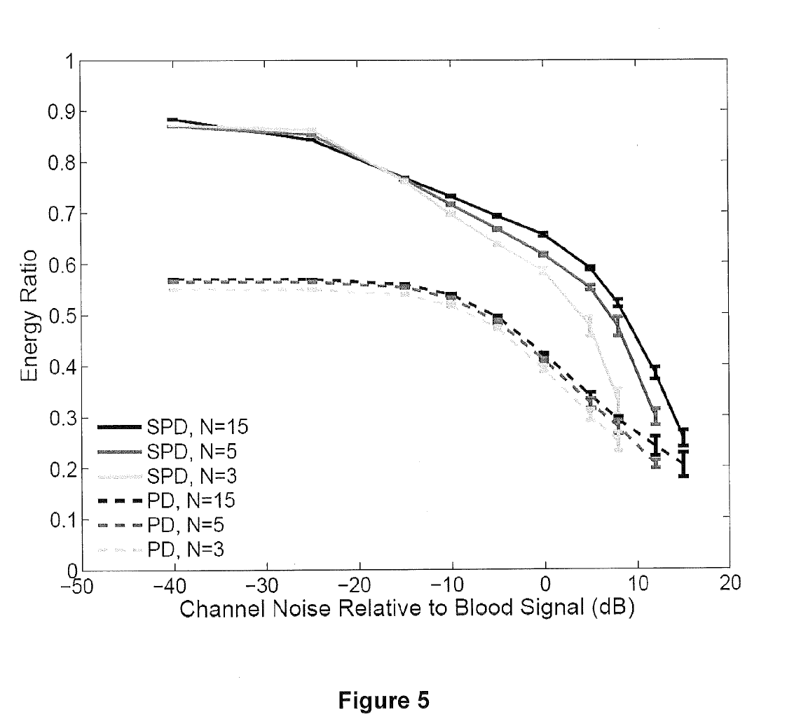

FIG. 5 shows the energy ratio calculations for both the simulated PD and SPD images as a function of channel noise. The energy ratios of the SPD images are significantly greater than that of the PD images. The energy ratio indicates that the SPD images demonstrate greater and smoother fill within the vessel region. The energy ratio is not significantly impacted by the ensemble length, however, except at the larger noise values. The energy ratio is improved for the larger ensemble sizes in this region because there is greater correlation of the blood signal across the ensemble (due to the changing f_{prf}).

If the blood signal is correlated, then the sum of the blood signal's power increases at a faster rate than the noise. The blood signal is likely not correlated over the entire ensemble; however, the additional independent samples of blood signal will contribute to a smoother profile, much in the way that spatial compounding generates a smoother speckle pattern in B-mode imaging.

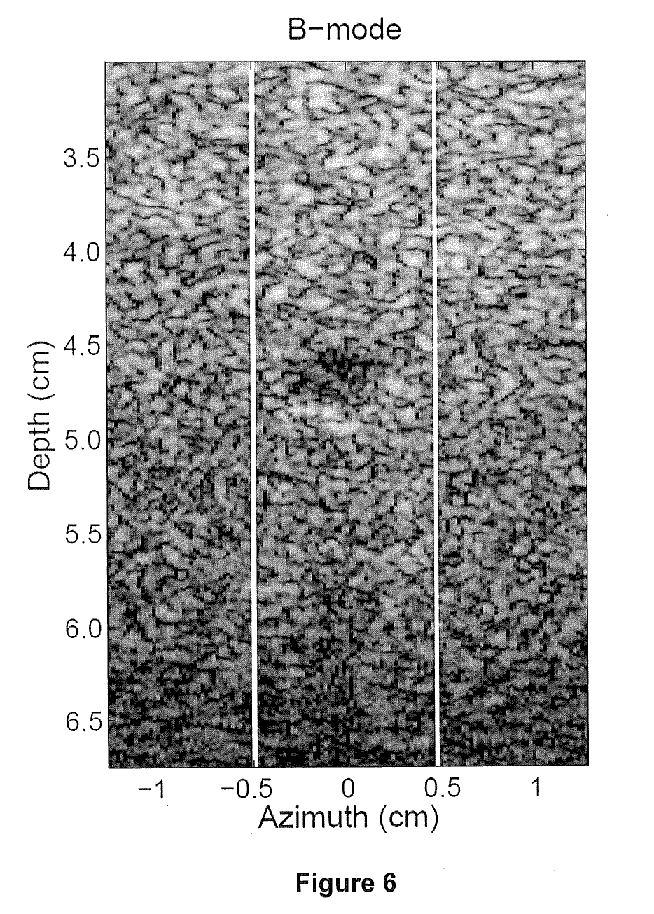

FIG. 6 shows a B-mode image of the flow phantom. A cross-section of a 4 mm vessel can be observed at approximately 4.75 cm depth. The vessel does not appear fully anechoic because the cornstarch and water mixture can produce a stronger backscatter than blood, depending on the concentration of the cornstarch. The white lines in this image demarcate the region where ensemble data was acquired for flow detection.

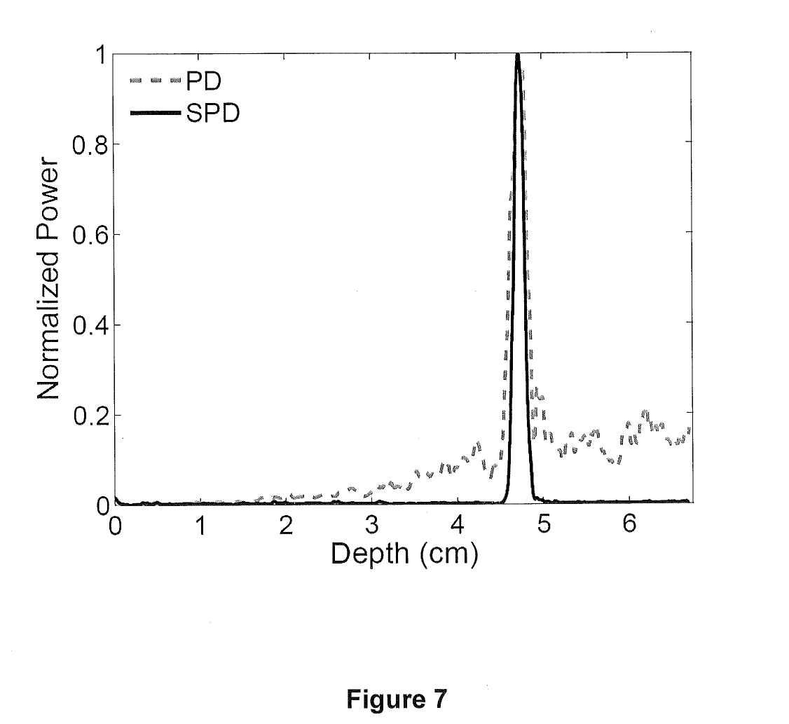

FIG. 7 is a plot of PD and SPD values through the center of the vessel in the flow phantom, normalized by the maximum power value. The PD and SPD images were created with an ensemble size of 15. The center line of the PD image (dashed line) displays a clear indication of flow in a vessel at 4.75 cm. Thermal noise is visible in this plot as the increasing baseline value with depth. The thermal noise increases because the ultrasound signal is attenuated with depth, thereby decreasing the signal-to-noise ratio of the Doppler signal of the deeper signals. The center line of the SPD image also shows a clear indication of flow in a vessel at 4.75 cm. The SPD image shows no change in the baseline value, however, which remains at approximately 0 throughout depth. The lack of change in this baseline value occurs because the SPD image is insensitive to the thermal noise. The thermal noise is spatially incoherent, and therefore the SLSC processing suppresses its image value.

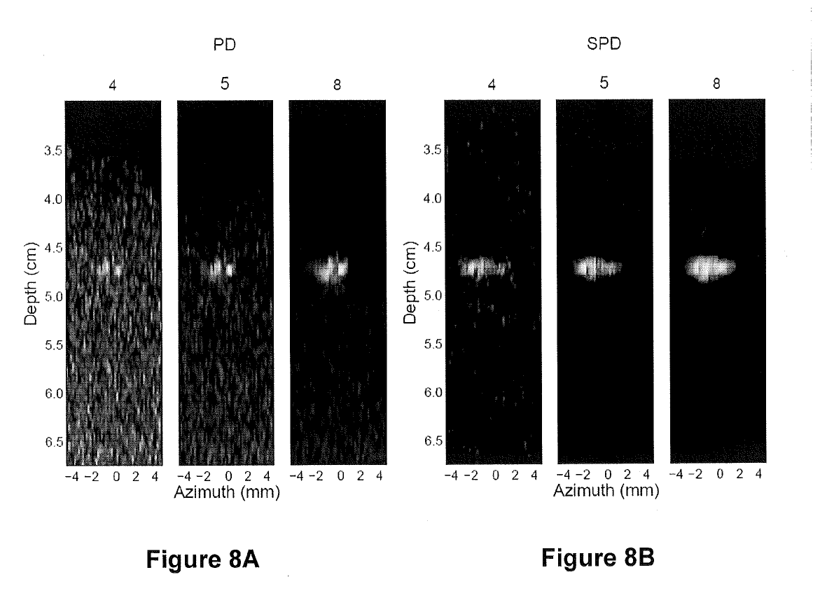

FIG. 8A is a PD image and FIG. 8B is an SPD image of the vessel phantom with ensemble sizes that are smaller than is used conventionally. All images are logarithmically compressed and show 30 dB of dynamic range, except the images with ensembles of 4, which show 34 dB of dynamic range. The dynamic range limitation is used to threshold the PD signal in order to display expected flow and suppress unwanted thermal noise. In the case of the PD images (FIG. 8A), the low ensemble size of 4 is incapable of separating the flow signal from the thermal noise. The visual detection of flow improves at an ensemble of 5, although thermal noise is still visible. At an ensemble of 8, a sufficient number of samples are utilized to separate the flow signal and the background noise.

In the SPD images (FIG. 8B), the flow in the vessel is clearly visible for all ensemble sizes. The area of the flow visualization is slightly smaller for the ensemble size of 4 compared to the other ensemble sizes. The significance of this result is that, assuming computational complexity does not impact the frame rate, power Doppler imaging can be performed at faster frame rates with less degrading effects from noise. The energy ratios of the flow in these images are shown in the Table below, and show an increasing energy ratio with increasing ensemble size for both PD and SPD imaging. Like the simulated images, the SPD images show a significantly greater energy ratio than the PD images. Neither the PD nor the SPD images display flow over the entire vessel. This is likely a result of clumping of the cornstarch from the slow flow rate.

TABLE 1
Energy Ratio in Flow Phantom

| | |
|---|---|
| Ensemble | Energy Ratio |

| Length | PD | SPD |
|---|---|---|
| 4 | 0.18 | 0.32 |
| 5 | 0.28 | 0.50 |
| 8 | 0.40 | 0.64 |

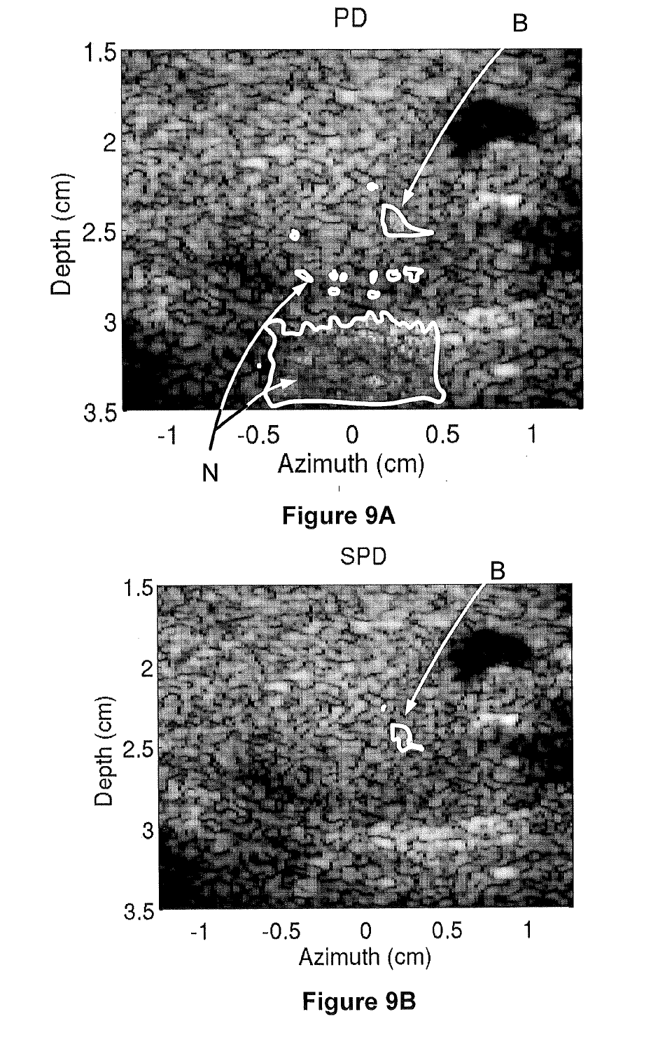

FIG. 9A is a PD image and FIG. 9B is an SPD image of an in vivo thyroid. The PD and SPD images of FIGS. 9A-9B were formed using an ensemble size of 5 and are overlayed on the B-mode image. The PD and SPD images are showing a vessel within the thyroid that makes a branch or bend at nearly 90 degrees, and was confirmed by observing the vessel with Doppler imaging on a clinical scanner. The blood B is shown at approximately 2 cm depth and may be indicated with a color overlay. The irregular shape or “leg” is the corner or branching of the vessel. The display threshold of the PD image has been adjusted such that the PD image shows the vessel approximately the same as in the SPD image. The PD image displays more thermal noise N in the lower part of the image, although this is may be undetected in clinical systems where the field-of-view is often restricted to a box or range. The PD image also shows numerous artifacts at approximately 2.75 cm depth. These artifacts can be reduced by increasing the threshold of the PD image, but this decreases the visualization of the flow, and eliminates the flow detected at the corner or branching vessel.

Accordingly, flow imaging techniques based on the spatial coherence of backscatter from blood are described. In some embodiments, thermal noise, which can obscure slow flow in power Doppler imaging when the noise is of similar amplitude to the blood signal, may be reduced. In some embodiments, lower ensemble lengths may be used, which can significantly improve the poor frame rates associated with Doppler imaging techniques.

The foregoing is illustrative of the present invention and is not to be construed as limiting thereof. Although a few example embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method for imaging fluid in a sample, the method comprising:
acquiring a plurality of echo signals for a region of interest in the sample from a plurality of ultrasound transducer elements in an ultrasound transducer array;
applying a stationary echo cancellation to the plurality of echo signals to reduce or remove echo signals that are associated with stationary and/or slower moving features to provide a corresponding plurality of filtered signals;
extracting coherence information from the plurality of filtered signals; and
imaging the region of interest in response to the coherence information from the plurality of filtered signals.

2. The method of claim 1, wherein the plurality of echo signals are responsive to time-delayed interrogation signals from the ultrasound transducer elements.

3. The method of claim 1, wherein the plurality of echo signals are responsive to a laser acoustic excitation.

4. The method of claim 1, wherein the plurality of echo signals are beamformed or partially beamformed echo signals received from a location in the region of interest.

5. The method of claim 4, wherein the plurality of echo signals are beformed or partially beamformed by a delay-and-sum beamformer and/or a frequency domain beamformer.

6. The method of claim 1, wherein applying a stationary echo cancellation comprises applying a highpass or eigendecomposition filter to the plurality of time-delayed echo signals.

7. The method of claim 1, wherein extracting coherence information from the plurality of filtered signals comprises computing correlation coefficients, covariance normalization, cross correlation coefficients, and/or cosine of the phase difference of filtered signals for the plurality of filtered signals.

8. The method of claim 1, further comprising storing the plurality of time-delayed echo signals and then applying the stationary echo cancellation to the plurality of time-delayed echo signals to reduce or remove echo signals that are associated with stationary and/or slower moving features to provide a corresponding plurality of filtered signals.

9. The method of claim 1, wherein imaging the region of interest in response to the coherence information from the plurality of filtered signals comprises overlaying the coherence information on an image.

10. A system for imaging fluid in a sample, the system comprising:
an ultrasound transducer array having a plurality of ultrasound transducer elements, the ultrasound transducer array being configured to acquire a plurality of echo signals for a region of interest in the sample from the plurality of ultrasound transducer elements in the ultrasound transducer array; and
a controller comprising:
a stationary echo cancellation unit configured to apply a stationary echo cancellation to the plurality of echo signals to reduce or remove echo signals that are associated with stationary and/or slower moving features to provide a corresponding plurality of filtered signals, and
a coherence information unit configured to extract coherence information from the plurality of filtered signals and to image the region of interest in response to the coherence information from the plurality of filtered signals.

11. The system of claim 10, wherein the echo signals are responsive to time-delayed interrogation signals from the ultrasound transducer elements.

12. The system of claim 10, further comprising a laser unit configured to excite the region of interest such that the echo signals are responsive to a laser acoustic excitation.

13. The system of claim 10, further comprising a beamformer that is configured to beamform or partially beamform the plurality of echo signals received from a location in the region of interest.

14. The system of claim 13, wherein the beamformer applies a delay-and-sum beamformer and/or a frequency domain beamformer to beamform or partially beamform the plurality of echo signals.

15. The system of claim 10, wherein the stationary echo cancellation unit is configured to apply the stationary echo cancellation by applying a highpass or eigendecomposition filter to the plurality of time-delayed echo signals.

16. The system of claim 10, wherein the coherence information unit is configured to extract coherence information from the plurality of filtered signals by computing correlation coefficients, covariance normalization, cross correlation coefficients, and/or cosine of the phase difference of filtered signals for the plurality of filtered signals.

17. The system of claim 10, wherein the controller comprises a channel acquisition unit configured to store the plurality of time-delayed echo signals.

18. The system of claim 10, wherein the coherence information unit is configured to image the region of interest in response to the coherence information from the plurality of filtered signals by overlaying the coherence information on an image.

19. A computer program product for imaging fluid in a sample, the computer program product comprising a non-transient computer readable medium having computer readable program code embodied therein, the computer readable program code comprising:
computer readable program code configured to acquire a plurality of time-delayed echo signals for a region of interest in the sample from a plurality of ultrasound transducer elements in an ultrasound transducer array;
computer readable program code configured to apply a stationary echo cancellation to the plurality of time-delayed echo signals to reduce or remove echo signals that are associated with stationary and/or slower moving features to provide a corresponding plurality of filtered signals;
computer readable program code configured to extract coherence information from the plurality of filtered signals; and
computer readable program code configured to image the region of interest in response to the coherence information from the plurality of filtered signals.

20. The computer program product of claim 19, wherein the time-delayed echo signals are responsive to time-delayed interrogation signals from the ultrasound transducer elements.

21. The computer program product of claim 19, wherein the time-delayed echo signals are responsive to a laser acoustic excitation.

22. The computer program product of claim 19, further comprising computer readable program code configured to beamform or partially beamform the plurality of echo signals received from a location in the region of interest.

23. The computer program product of claim 22, wherein the computer readable program code configured to beamform or partially beamform the plurality of echo signals is further configured to apply a delay-and-sum beamformer and/or a frequency domain beamformer.

24. The computer program product of claim 19, wherein the computer readable program code configured to apply a stationary echo cancellation comprises computer readable program code configured to apply a highpass or eigendecomposition filter to the plurality of time-delayed echo signals.

25. The computer program product of claim 19, wherein the computer readable program code configured to extract coherence information from the plurality of filtered signals comprises computer readable program code configured to compute correlation coefficients, covariance normalization, cross correlation coefficients, and/or cosine of the phase difference of filtered signals for the plurality of filtered signals.

26. The computer program product of claim 19, further comprising computer readable program code configured to store the plurality of time-delayed echo signals and then apply the stationary echo cancellation to the plurality of time-delayed echo signals to reduce or remove echo signals that are associated with stationary and/or slower moving features to provide a corresponding plurality of filtered signals.

27. The computer program product of claim 19, wherein the computer readable program code configured to image the region of interest in response to the coherence information from the plurality of filtered signals comprises computer readable program code configured to overlay the coherence information on an image.
